(19) United States
(12) Patent Application Publication
(10) Pub. No.: US 2025/0253011 A1
(43) Pub. Date: Aug. 7, 2025

(54) TECHNIQUES FOR IMPROVED TUMOR MUTATIONAL BURDEN (TMB) DETERMINATION USING A POPULATION-SPECIFIC GENOMIC REFERENCE

(71) Applicant: Seven Bridges Genomics Inc., Charlestown, MA (US)

(72) Inventors: Amit Jain, London (GB); Özem Kalay, Istanbul (TR); Elif Arslan, Ankara (TR)

(21) Appl. No.: 19/029,467

(22) Filed: Jan. 17, 2025

Related U.S. Application Data
(60) Provisional application No. 63/549,097, filed on Feb. 2, 2024.

(51) Int. Cl. G16B 40/20; G16B 30/00; G16H 10/40

(52) U.S. Cl. CPC G16B 40/20; G16B 30/00; G16H 10/40

(57) ABSTRACT

Described herein are techniques for determining tumor mutational burden (TMB) of a tumor sample previously obtained from a subject. In some embodiments, the techniques include obtaining sequence reads, the sequence reads having been previously obtained by sequencing the tumor sample; aligning the sequence reads to a population-specific genomic reference graph representing a linear reference sequence and population-specific variants relative to the linear reference sequence, wherein the population-specific variants are variants associated with at least one population to which the subject belongs; identifying, based on a result of aligning the sequence reads to the population-specific genomic reference graph, a plurality of somatic variants; and determining the TMB of the tumor sample using the identified plurality of somatic variants.

### CROSS-REFERENCE TO RELATED APPLICATIONS

This application claims the benefit of priority under 35 U.S.C. § 119 (e) of U.S. Provisional Patent Application No. 63/549,097, titled “TECHNIQUES FOR IMPROVED TUMOR MUTATIONAL BURDEN (TMB) DETERMINATION USING A POPULATION-SPECIFIC GENOMIC REFERENCE,” filed Feb. 2, 2024, which is incorporated by reference herein in its entirety.

### BACKGROUND

Tumor mutation burden (TMB) is a measure used in the field of oncology to quantify a number of variants within a tumor's genome. In some embodiments, TMB is determined by identifying a total number of non-synonymous, somatic variants per megabase (Mb) of genome examined. Non-synonymous variants are variants that lead to a change in the amino acid sequence of a protein. This has been recognized as a potential biomarker for predicting response to immune checkpoint inhibitors, a type of immunotherapy.

### BRIEF SUMMARY

Some aspects provide for a method for determining tumor mutational burden (TMB) of a tumor sample previously obtained from a subject, the method comprising: using at least one computer hardware processor to perform: obtaining sequence reads, the sequence reads having been previously obtained by sequencing the tumor sample; aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges; identifying, using results of aligning the sequence reads to the population-specific genomic reference graph, a plurality of somatic variants; and determining the TMB of the tumor sample using the identified plurality of somatic variants.

Some aspects provide for a system, comprising: at least one computer hardware processor; and at least one non-transitory computer-readable storage medium storing processor-executable instructions that, when executed by at least one computer hardware processor, causes the at least one computer hardware processor to perform a method for determining tumor mutational burden (TMB) of a tumor sample previously obtained from a subject, the method comprising: obtaining sequence reads, the sequence reads having been previously obtained by sequencing the tumor sample; aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges; identifying, using results of aligning the sequence reads to the population-specific genomic reference graph, a plurality of somatic variants; and determining the TMB of the tumor sample using the identified plurality of somatic variants.

Some aspects provide for at least one non-transitory computer-readable storage medium storing processor-executable instructions that, when executed by at least one computer hardware processor, causes the at least one computer hardware processor to perform a method for determining tumor mutational burden (TMB) of a tumor sample previously obtained from a subject, the method comprising: obtaining sequence reads, the sequence reads having been previously obtained by sequencing the tumor sample; aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges; identifying, using results of aligning the sequence reads to the population-specific genomic reference graph, a plurality of somatic variants; and determining the TMB of the tumor sample using the identified plurality of somatic variants.

Embodiments of any of the above aspects may have one or more of the following features.

Some embodiments further comprise: determining, using the determined TMB, to administer an immunotherapy to the subject.

In some embodiments, determining to administer the immunotherapy to the subject comprises: determining whether the determined TMB is greater than or equal to a threshold TMB; and determining to administer the immunotherapy to the subject after determining that the determined TMB is greater than or equal to the threshold TMB.

In some embodiments, the sequence reads were previously-obtained by sequencing the tumor sample using whole exome sequencing, and the threshold TMB is between 150 variants/megabase (Mb) and 200 variants/Mb.

Some embodiments further comprise: performing whole exome sequencing of the tumor sample to obtain the sequence reads.

In some embodiments, the sequence reads were previously obtained by sequencing the tumor sample using whole genome sequencing, and the threshold TMB is between 5 variants/Mb and 15 variants/Mb.

Some embodiments further comprise: performing whole genome sequencing of the tumor sample to obtain the sequence reads.

Some embodiments further comprise: administering the immunotherapy to the subject.

In some embodiments, the immunotherapy is an immune checkpoint inhibitor.

In some embodiments, the immune checkpoint inhibitor is pembrolizumab.

In some embodiments, determining the TMB of the tumor sample comprises: determining a number of somatic variants included in the identified plurality of somatic variants; determining a size of a genomic region sequenced during the sequencing of the tumor sample; and determining a ratio of the number of somatic variants to the size of the genomic region sequenced during the sequencing of the tumor sample.

In some embodiments, identifying the plurality of somatic variants comprises: identifying a plurality of candidate variants using results of aligning the sequence reads to the population-specific genomic reference graph; and filtering the plurality of candidate variants using at least a portion of the population-specific genomic reference graph to obtain the plurality of somatic variants.

In some embodiments, filtering the plurality of candidate variants using at least the portion of the population-specific genomic reference graph to obtain the plurality of somatic variants comprises: identifying, using at least the portion of the population-specific genomic reference graph, one or more germline variants from among the plurality of candidate variants; and excluding the one or more germline variants from the plurality of somatic variants.

In some embodiments, the results of aligning the sequence reads to the population-specific genomic reference graph comprise a plurality of aligned sequence reads, and wherein identifying the plurality of somatic variants comprises: providing, as input to a somatic variant caller, the plurality of the aligned sequence reads and at least a portion of the population-specific genomic reference graph; and obtaining, as output from the somatic variant caller, the plurality of somatic variants.

Some embodiments further comprise: generating the population-specific genomic reference graph, the generating comprising: obtaining an initial genomic reference, the initial genomic reference including the linear reference sequence; and augmenting the initial genomic reference with the population-specific variants.

In some embodiments, augmenting the initial genomic reference with the population-specific variants comprises augmenting the initial genomic reference with one or more nodes and one or more edges, the one or more nodes and the one or more edges representing at least some of the population-specific variants.

In some embodiments, the population-specific genomic reference graph represents at least a portion of a human genome.

In some embodiments, the population-specific genomic reference graph represents at least a chromosome of the human genome.

In some embodiments, the population-specific genomic reference graph represents at least 10,000,000 nucleotides, at least 50,000,000 nucleotides, at least 100,000,000 nucleotides, at least 150,000,000 nucleotides, at least 200,000,000 nucleotides, or at least 250,000,000 nucleotides.

In some embodiments, the population-specific genomic reference graph is a directed acyclic graph (DAG).

In some embodiments, the nodes representing nucleotide sequences stored as respective strings of one or more symbols, and the edges including an edge representing a connection between at least two of the nodes.

In some embodiments, the at least one data structure comprises objects representing the nodes and pointers representing the edges, the objects comprising a first object representing a first node of the nodes, the first object storing at least one pointer representing at least one edge in the population-specific genomic reference graph from the first node to at least one other node.

Some embodiments further comprise sequencing the tumor sample to obtain the sequence reads.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for determining the tumor mutational burden (TMB) of a tumor sample from a subject, according to some embodiments of the technology described herein.

FIG. 1C is a diagram of an illustrative technique for obtaining population-specific germline variants used to filter out germline variants to identify somatic variants, according to some embodiments of the technology described herein.

FIG. 2 is a block diagram of an example system 200 for determining the TMB of a tumor sample from a subject, according to some embodiments of the technology described herein.

FIG. 3 is a flowchart of an illustrative process 300 for determining the TMB of a tumor sample from a subject, according to some embodiments of the technology described herein.

FIG. 4 is a graph showing the fraction of individual genetic variation present in a population-specific genomic reference graph, according to some embodiments of the technology described herein.

FIG. 5 is a graph showing that determining the TMB, in accordance with embodiments of the technology described herein, is more accurate as compared to conventional techniques.

FIG. 6 is a graph showing that determining the TMB, in accordance with embodiments of the technology described herein, is more precise as compared to conventional techniques.

FIG. 7 is a schematic diagram of an illustrative computing device with which aspects described herein may be implemented.

### DETAILED DESCRIPTION

The inventors have developed techniques for determining tumor mutational burden (TMB) of a tumor sample from a subject. In some embodiments, the techniques for determining TMB include (a) obtaining sequence reads from the tumor sample, (b) aligning the sequence reads to a population-specific genomic reference graph, (c) identifying somatic variants based on results of aligning the sequence reads to the population-specific genomic reference graph, and (d) determining the TMB for the tumor sample based on the identified somatic variants. In some embodiments, the TMB may be used to identify a therapy to be administered to the subject.

Tumor mutation burden (TMB) is a measure used in to quantify a number of variants within a tumor's genome. Accurately estimating TMB for a tumor sample relies on accurately identifying somatic variants in the tumor sample, while disregarding the germline variants that characterize the subject's genome.

Some conventional techniques distinguish between somatic and germline variants by simultaneously analyzing a tumor sample from a subject and a non-tumor sample from the subject and filtering out variants which are present in both samples (e.g., germline variants). However, there are several drawbacks associated with such conventional techniques. First, due to the complexities of analyzing multiple samples simultaneously, estimating TMB using the conventional techniques is unreliable. There are various factors that may adversely affect the data analysis, which contribute to its unreliability. Such factors include, for example, tumor heterogeneity, artifacts related to tissue preparation, and discrepancies across sequencing assays. Second, it is burdensome to obtain both tumor and non-tumor samples from a subject. Obtaining samples is an invasive procedure and should be minimized or avoided where possible.

Other conventional techniques distinguish between somatic and germline variants by using public databases of germline variants. Variants identified as being both present in the tumor sample from the subject and present in the public databases are filtered out, isolating the somatic variants present in the tumor sample. This strategy suffers from the drawback that public databases lack genomic information about diverse populations and ancestries (e.g., African, Asian, etc.) due to the historical acceptance of European donors' genome as representative of the human genome. As a result, conventional techniques which employ public databases of germline variants fail to adequately filter out germline variants in genomes of individuals belonging to non-European populations. Due to the significant proportion of germline variants being misclassified as somatic variants, TMB estimates are inflated and are therefore unreliable for use in predicting a subject's therapeutic response, especially for individuals belonging to non-European populations.

Accordingly, the inventors have developed techniques that address the above-described challenges associated with the conventional techniques for determining TMB for a tumor sample from a subject. In some embodiments, the techniques include: (a) obtaining sequence reads previously obtained from the tumor sample, (b) aligning the sequence reads to a population-specific genomic reference graph, (c) identifying somatic variants based on results of aligning the sequence reads to the population-specific genomic reference graph, and (d) determining the TMB using the identified somatic variants. In some embodiments, the population-specific genomic reference graph represents variants that are common among one or more populations to which the subject belongs. By using such a population-specific genomic reference graph, the techniques developed by the inventors more accurately and precisely detect germline variants in the subject's genome, without requiring that an additional non-tumor sample be obtained from the subject and analyzed in parallel with the tumor sample.

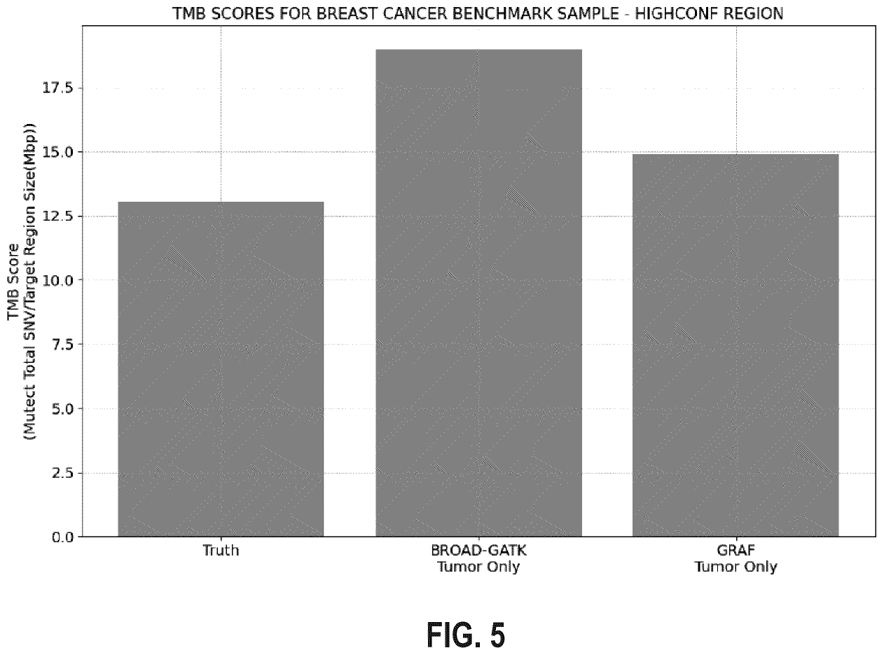

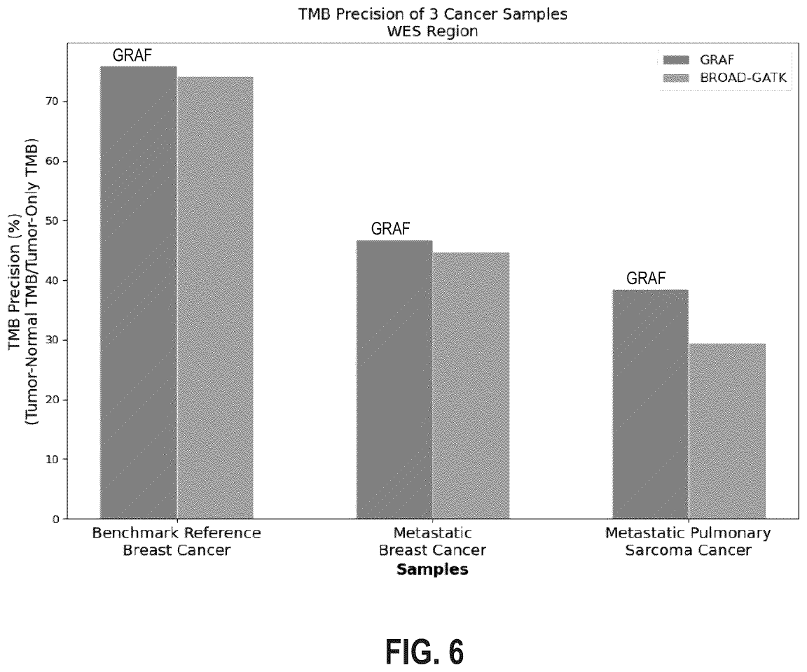

By more accurately and precisely distinguishing between somatic and germline variants in a tumor sample, the techniques developed by the inventors also increase the accuracy and precision of the TMB estimate determined using the somatic variants identified for the tumor sample. The improved accuracy and precision are demonstrated in FIG. 5 and FIG. 6, which show the results of comparing TMB estimates determined according to the techniques developed by the inventors (“GRAF”) to TMB estimates determined according to the conventional techniques (“GATK-BROAD”). The conventional techniques do not involve the use of a population-specific genomic reference for distinguishing between somatic and germline variants and determining TMB. As shown in FIG. 5, the GRAF techniques yielded a TMB value that is better aligned with the “true” TMB value of a benchmark sample. As shown in FIG. 6, the GRAF techniques led to more precise TMB scores across three unrelated samples extracted from the biopsies of different cancer types.

The accuracy and precision of TMB values have important therapeutic implications. TMB values can be used to predict how a subject will respond to a particular therapy. For example, a high TMB value (e.g., above a threshold) may indicate that a patient will have a positive therapeutic response to an immune checkpoint inhibitor (ICI) such as pembrolizumab. By contrast, the same therapy may result in serious side effects and be contraindicated for subjects with a low TMB value (e.g., below a threshold). By accurately and precisely determining TMB for a tumor sample for a subject, the techniques developed by the inventors can be used to accurately predict therapeutic response, to administer therapies that will be benefit subjects, and to avoid administering therapies that will result in serious side effects.

Following below are descriptions of various concepts related to, and embodiments of, techniques for determining TMB for a tumor sample from a subject. It should be appreciated that various aspects described herein may be implemented in any of numerous ways, as techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

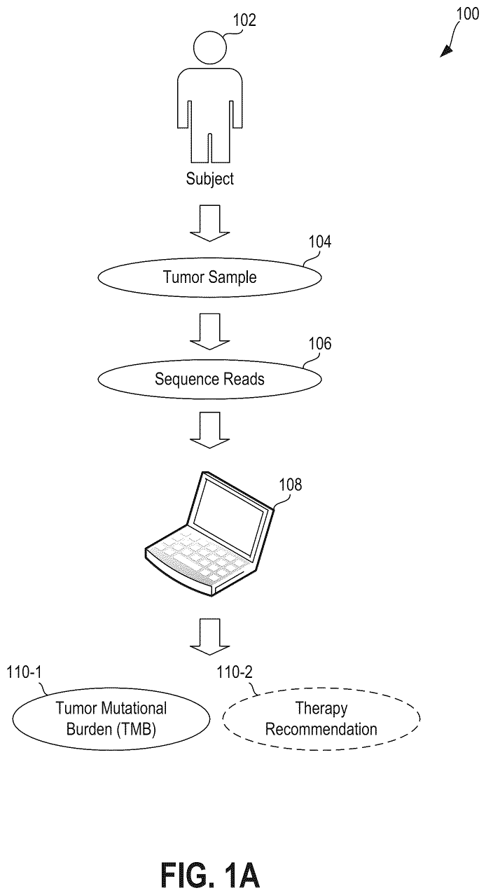

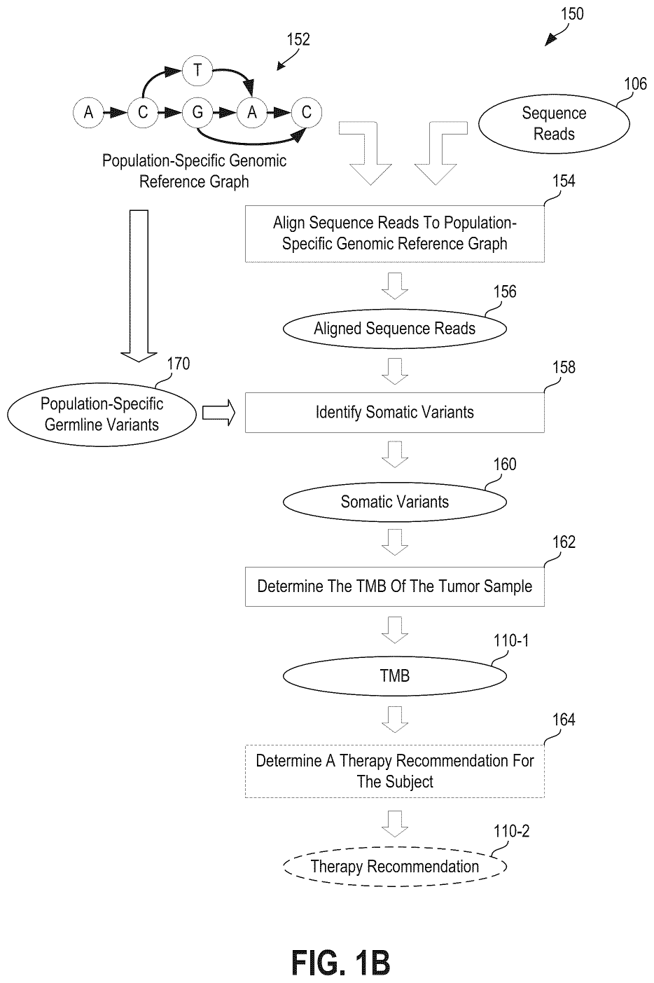

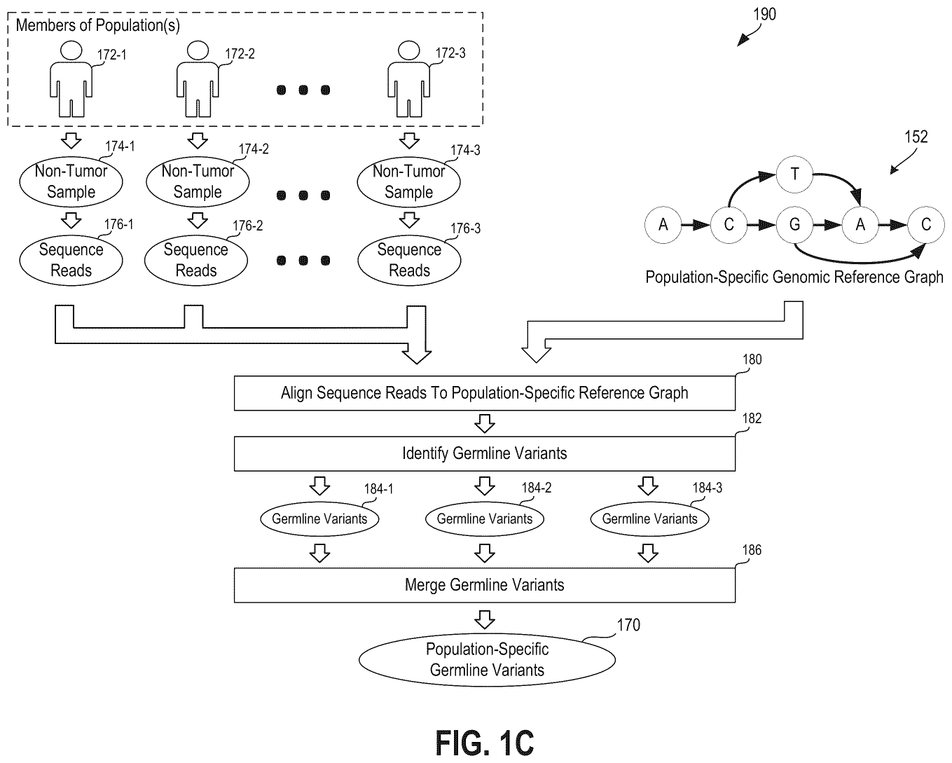

FIG. 1A is a diagram of an illustrative technique 100 for determining the tumor mutational burden (TMB) of a tumor sample from a subject, according to some embodiments of the technology described herein. Technique 100 includes obtaining sequence reads 106 from a tumor sample 104 previously obtained from subject 102 and processing the sequence reads 106 using computing device 108 to obtain the TMB 110-1 of the tumor sample 104 and/or a therapy recommendation 110-2 for the subject 102. For example, the computing device 108 may use the TMB 110-1 to determine the therapy recommendation 110-2.

In some embodiments, aspects of the illustrated technique 100 may be implemented in a clinical or laboratory setting. For example, aspects of the illustrated technique 100 may be implemented on a computing device 108 that is located within the clinical or laboratory setting. In some embodiments, the computing device 108 may obtain sequence reads 106 from a sequencing platform co-located with the computing device 108 within the clinical or laboratory setting. For example, the computing device 108 may be included within the sequencing platform. In some embodiments, the computing device 108 may indirectly obtain the sequence reads 106 from a sequencing platform that is located externally from or co-located with the computing device 108 within the clinical or laboratory setting. For example, the computing device 108 may obtain the sequence reads 106 via at least one communication network, such as the Internet or any other suitable communication network(s), as aspects of the technology described herein are not limited in this respect.

In some embodiments, aspects of the illustrative technique 100 may be implemented in a setting that is located externally from a clinical or laboratory setting. In this case, the computing device 108 may indirectly obtain sequence reads 106 from a sequencing platform located within or externally to a clinical or laboratory setting. For example, the sequence reads 106 may be provided to the computing device 108 via at least one communication network, such as the Internet or any other suitable communication network(s), as aspects of the technology described herein are not limited in this respect.

As shown in FIG. 1A, sequence reads 106 are obtained by processing a tumor sample 104 obtained from the subject 102. A tumor sample, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells. The origin, type, or preparation methods of the tumor sample 104 may include any of the embodiments relating to tumor samples described in the section “Biological Samples.”

In some embodiments, the sequence reads 106 are obtained using a sequencing platform such as a next generation sequencing platform (e.g., Illumina®, Roche®, Ion Torrent®, etc.), or any high-throughput or massively parallel sequencing platform. In some embodiments, the sequence reads 106 may be the result of non-next generation sequencing (e.g., Sanger sequencing).

The sequence reads 106 may include DNA sequence reads, DNA exome sequence reads (e.g., reads obtained from whole exome sequencing (WES)), DNA genome sequence reads (e.g., reads obtained from whole genome sequencing (WGS)), gene sequence reads, bias-corrected sequence reads, or any other suitable type of sequence reads obtained from a sequencing platform and/or derived from data obtained from a sequencing platform. The origin, type, or preparation methods of the sequence reads may include any of the embodiments described in the section “Sequencing Data.”

In some embodiments, the computing device 108 is used to process the sequence reads 106 to determine the TMB 110-1 of the tumor sample 104 and/or a therapy recommendation 110-2 for the subject 102. The computing device 108 may be operated by a user such as a doctor, clinician, researcher, the subject 102, and/or any other suitable entity. For example, the user may provide the sequence reads 106 as input to the computing device 108 (e.g., by uploading a file), provide user input specifying processing or other methods to be performed using the sequence reads 106, and/or provide input specifying one or more clinical features associated the subject 102 and/or the tumor sample 104.

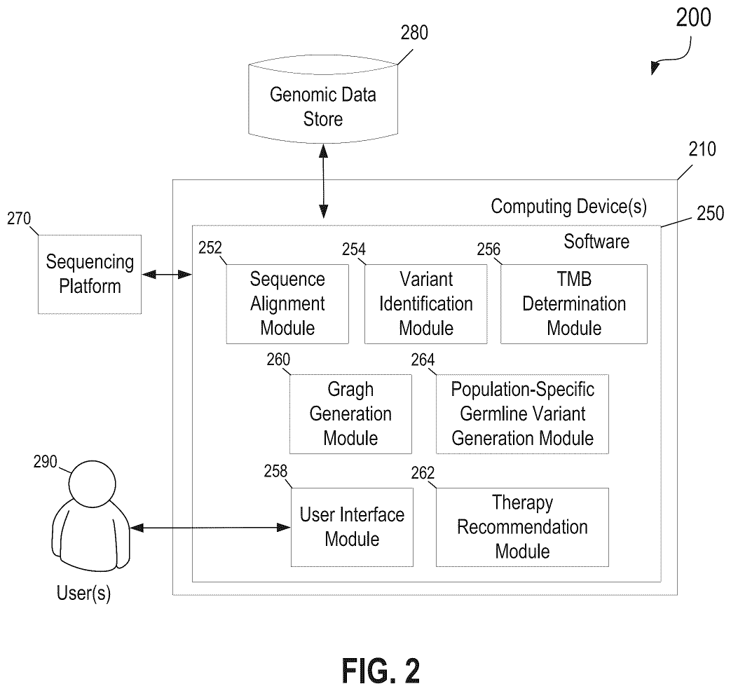

In some embodiments, software on the computing device 108 may be used to determine the TMB 110-1 for the tumor sample 104 and/or to determine a therapy recommendation 110-2 for the subject 102. An example of computing device 108 and such software is described herein including at least with respect to FIG. 2 (e.g., computing device(s) 210 and software 250).

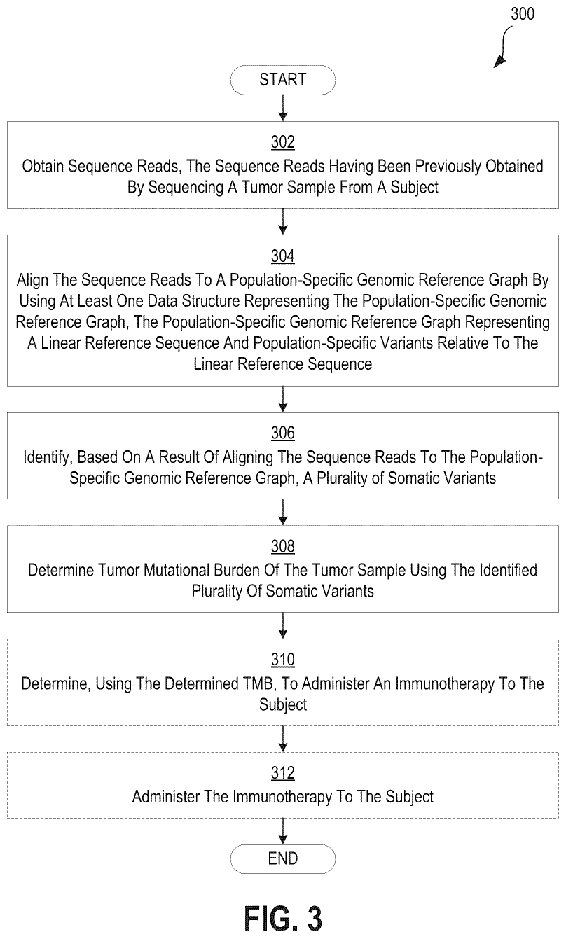

In some embodiments, software on the computing device 108 may be configured to process at least some (e.g., all) of the sequence reads 106 to determine the TMB 110-1. In some embodiments, this may include: (a) aligning the sequence reads to a population-specific genomic reference graph representing a linear reference sequence and population-specific variants relative to the linear reference sequence, (b) identifying, based on a result of aligning the sequence reads to the population-specific genomic reference graph, a plurality of somatic variants; and (c) determining the TMB of the tumor sample using the identified plurality of somatic variants. Example techniques for determining the TMB for a tumor sample are described herein including at least with respect to FIG. 1B and FIG. 3.

In some embodiments, software on the computing device 108 may additionally, or alternatively, determine a therapy recommendation 110-2 for the subject 102. For example, the therapy recommendation 110-2 may identify one or more immunotherapies recommended for treating the subject. Additionally, or alternatively, the therapy recommendation 110-2 may prompt a user (e.g., a doctor, a clinician, etc.) to administer a recommended therapy to the subject 102. Additionally, or alternatively, the therapy recommendation 110-2 may identify one or more therapies that are not recommended for treating the subject. For example, such a therapy may be predicted to result in side effects and/or be contraindicated for the subject 102.

In some embodiments, the computing device 108 is configured to generate an output indicating the TMB 110-1 and/or the therapy recommendation 110-2. In some embodiments, the output of the computing device 108 is stored (e.g., in memory), displayed via a user interface, transmitted to one or more other devices, used to generate a report, or otherwise processed using any other suitable techniques, as aspects of the technology described herein are not limited in this respect. For example, the output of the computing device 108 may be displayed via a graphical user interface (GUI) of a computing device (e.g., computing device 108).

In some embodiments, the output of the computing device 108 may be in the form of a report, such as a report including an indication of TMB 110-1 determined for the tumor sample 104 and/or an indication of a therapy recommendation 110-2 for the subject 102. The generated report can provide a summary of information, so that a clinician can identify the TMB for the tumor sample 104 and/or a therapy to be administered to the subject 102. The report as described herein may be a paper report, an electronic record, or a report in any format that is deemed suitable in the art. The report may be shown and/or stored on a computing device known in the art (e.g., a handheld device, desktop computer, smart device, website, etc.). The report may be shown and/or stored on any device that is suitable as understood by a skilled person in the art.

In some embodiments, the methods and reports disclosed herein may include database management for the keeping of generated reports. For instance, the methods as disclosed herein can create a record in a database for the subject 102 and populate the specific record with data for the subject 102. In some embodiments, the generated report can be provided to the subject 102, clinicians, doctors, researchers, or any other suitable entity. In some embodiments, a network connection can be established to a server computer that includes the data and report for receiving or outputting. In some embodiments, the receiving and outputting of the data or report can be requested from the server computer.

In some embodiments, the computing device 108 includes one or multiple computing devices. In some embodiments, when the computing device 108 includes multiple computing devices, each of the computing devices may be used to perform the same process or processes. For example, each of the multiple computing devices may include software used to implement process 300 shown in FIG. 3. In some embodiments, when the computing device 108 includes multiple computing devices, the computing devices may be used to perform different processes or different aspects of a process. For example, one computing device may include software used to align sequence reads to a reference data structure (e.g., a population-specific genomic reference graph, etc.), while a different computing device may include software used to identify variants based on aligning the sequence reads to the reference data structure.

In some embodiments, when the computing device 108 includes multiple computing devices, the multiple computing devices may be configured to communicate via at least one communication network such as the Internet or any other suitable communication network(s), as aspects of the technology described herein are not limited in this respect. For example, one computing device may be configured to align sequence reads to a reference data structure, and then provide results of the alignment to one or more other computing devices via the communication network.

FIG. 1B is a diagram depicting an illustrative technique 150 for processing sequence reads 106 to determine TMB 110-1 of a tumor sample (e.g., tumor sample 104 shown in FIG. 1A) and/or to determine a therapy recommendation 110-2 for a subject (e.g., subject 102 shown in FIG. 1A). The illustrative techniques 150 includes (a) at act 154, aligning sequence reads 106 to the population-specific genomic reference graph 152 to obtain aligned sequence reads 156; (b) at act 158, identifying somatic variants 160 using the aligned sequence reads 156 and the population-specific germline variants 170; (c) at act 162, determining the TMB 110-1 of the tumor sample using the somatic variants 160; and (d) at act 164, determining a therapy recommendation 110-2 for the subject using the TMB 110-1. As described herein, including at least with respect to FIG. 1A, illustrative technique 150 may be implemented using a computing device such as computing device 108 shown in FIG. 1A.

As shown in FIG. 1B, illustrative technique 150 includes aligning sequence reads 106 to the population-specific genomic reference graph 152 at act 154. The population-specific genomic reference may represent a linear reference sequence and population-specific variants relative to the linear reference sequence. The linear reference sequence may include a human genome reference sequence such as, for example, human genome version 19 (hg19), hg38, Genome Reference Consortium human reference 38 (GRCh38), GRCh37, or any other suitable human genome reference sequence, as aspects of the technology described herein are not limited in this respect. The population-specific variants may represent variants that are common among members of one or more populations to which the subject belongs. Nonlimiting examples of populations include African ancestry (AFR), American ancestry (AMR), South-Asian ancestry (SAS), Eastern-Asian ancestry (EAS), and European ancestry (EUR). Variants that are specific to particular populations may be obtained from any suitable source such as, for example, the 1000 Genomes Project consortium. The population(s) to which the subject belongs may be identified using any suitable techniques, as aspects of the technology are not limited in this respect. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384), which is incorporated by reference herein in its entirety.

The population-specific genomic reference graph 152 may represent any suitable number of nucleotides, as aspects of the technology described herein are not limited in this respect. For example, the population-specific genomic reference graph may represent a number of nucleotides between 10 and 3 billion nucleotides, between 1,000 and 2 billion nucleotides, between 10,000 and 1 billion nucleotides, between 100,000 and 100 million nucleotides, between 1 million and 10 million nucleotides, or any other suitable number of nucleotides. Additionally, or alternatively, the population-specific genomic reference graph may represent at least 10, at least 100, at least 1,000, at least 10,000, at least 100,000, at least 1 million, at least 10 million, at least 50 million, at least 100 million, at least 150 million, at least 200 million, at least 250 million, or at least any other suitable number of nucleotides. Additionally, or alternatively, the population-specific genomic reference graph may represent at most 3 billion, at most 2 billion, at most 1 billion, at most 250 million, at most 150 million, at most 100 million, at most 50 million, at most 10 million, at most 1 million, or at most any other suitable number of nucleotides. It should be appreciated that any of the above-listed upper bounds may be coupled with any of the above-listed lower bounds.

In some embodiments, the sequence reads 106 may be aligned to the population-specific genomic reference graph 152 using any suitable graph alignment techniques, as aspects of the technology described herein are not limited in this respect. In some embodiments, the graph alignment may be performed using dynamic programming. In some embodiments, the graph alignment technique may include a linear alignment technique that has been modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”, each of which is incorporated by reference herein in its entirety.

In some embodiments, somatic variants 160 are identified, at act 158, using the aligned sequence reads 156 and the population-specific germline variants 170. In some embodiments, this is performed using somatic variant calling software. Nonlimiting examples of somatic variant calling software include Mutect2 software, rasm software, Strelka2 software, VarScan2 software, or any other suitable somatic variant calling software as aspects of the technology described herein are not limited in this respect. Mutect2software is described by Cibulskis, K., et al. (“Sensitive detection of somatic point mutations in impure and heterogeneous cancer samples.” Nature biotechnology 31.3 (2013): 213-219), which is incorporated by reference herein in its entirety. Strelka2 software is described by Saunders, C., et al. (“Strelka: accurate somatic small-variant calling from sequenced tumor-normal sample pairs.” Bioinformatics 28.14 (2012): 1811-1817), which is incorporated by reference herein in its entirety. VarScan2 software is described by Koboldt, D., et al. (“VarScan 2: somatic and copy number alteration discovery in cancer by exome sequencing.” Genome research 22.3 (2012): 568-576), which is incorporated by reference herein it its entirety.

In some embodiments, the somatic variants 160 identified, at act 158, include non-synonymous variants. Non-synonymous variants are variants that lead to a change in the amino acid sequence of a protein.

In some embodiments, the population-specific germline variants 170 (which may sometimes be referred to as a “panel of normals”) are used to identify the somatic variants. Population-specific germline variants may include germline variants that have been identified for one or more non-tumor samples (e.g., biological samples that are believed to have less than a threshold number of somatic variants). In some embodiments, the non-tumor samples are obtained, or were previously obtained, from members of the same one or more populations to which the subject (e.g., subject 102 shown in FIG. 1A) belongs. The population-specific germline variants may be used (e.g., used by somatic variant calling software) to distinguish between somatic variants and germline variants that are common among the members of the population(s), thereby resulting in a more accurate estimation of somatic variants for the subject. For example, if a variant is found in the population-specific germline variants, it may be filtered out and excluded from the final estimation of somatic variants.

The population-specific germline variants 170 may be obtained using any suitable techniques, as aspects of the technology described herein are not limited in this respect. In some embodiments, as shown in FIG. 1B, the population-specific germline variants 170 may be generated using the population-specific genomic reference graph 152. Example techniques for generating population-specific germline variants are described herein including at least with respect to FIG. 1C. In some embodiments, the population-specific germline variants may be obtained from a public database such as, for example, the public Genome Analysis Toolkit (GATK) population-specific germline variants.

In some embodiments, the TMB 110-1 of the tumor sample is determined, at act 162, using the somatic variants 160. In some embodiments, determining TMB may include determining the number of somatic variants in a defined region of the genome of the tumor sample. For example, in some embodiments, determining TMB may include determining the number of somatic variants per megabase (Mb). The size of the region of the genome of the tumor sample may depend on the assay used for sequencing the tumor sample. For example, whole genome sequencing covers the entire genome (e.g., including coding and non-coding regions of all genes) and whole exome sequencing (WES) covers the coding regions of all genes (e.g., thousands of genes). By contrast, smaller gene panels may cover coding regions of only some genes (e.g., hundreds of genes). Example techniques for determining TMB are described by Büttner, R., et al., (“Implementing TMB measurements in clinical practice: considerations on assay requirements.” ESMO open 4.1 (2019): e000442.), which is incorporated by reference herein in its entirety. Techniques for determining TMB are described herein including at least with respect to act 308 of process 300 shown in FIG. 3.

In some embodiments, the TMB 110-1 is used, at act 164, to determine a therapy recommendation 110-2 for the subject. In some embodiments, determining the therapy recommendation includes determining whether to administer a therapy to the subject. The therapy may include an immunotherapy such as an immune checkpoint inhibitor or any of the therapies described herein including at least in the section “Therapies.”

In some embodiments, determining whether to administer an immunotherapy to the subject includes determining whether the TMB 110-1 is greater than or equal to a threshold, and determining to administer the immunotherapy to the subject when the TMB 110-1 is greater than or equal to the threshold. In some embodiments, the threshold depends on the type of sequencing used to obtain the sequence reads. For example, when WGS is used to obtain the sequence reads, the threshold may be between 8 variants/Mb and 12 variants/Mb, between 9 variants/Mb and 11 variants/Mb, or any other suitable threshold. Additionally, or alternatively, when WGS is used to obtain the sequence reads, the threshold may be at least 8 variants/Mb, at least 9 variants/Mb, at least 10 variants/Mb, at least 11 variants/Mb, at least 12 variants/Mb, or at least any other suitable threshold. Additionally, or alternatively, when WGS is used to obtain the sequence reads, the threshold may be at most 8 variants/Mb, at most 9 variants/Mb, at most 10 variants/Mb, at most 11 variants/Mb, at most 12 variants/Mb, or at most any other suitable threshold. It should be appreciated that any of the above-listed upper bounds may be coupled with any of the above-listed lower bounds. In some embodiments, when WES is used to obtain the sequence reads, the threshold may be between 150 variants/Mb and 200 variants/Mb, between 160 and 190 variants/Mb, between 165 variants/Mb and 185 variants/Mb, between 170 variants/Mb and 180 variants/Mb, or any other suitable threshold. Additionally, or alternatively, when WES is used to obtain the sequence reads, the threshold may be at least 150 variants/Mb, at least 160 variants/Mb, at least 165 variants/Mb, at least 170 variants/Mb, at least 175 variants/Mb, at least 180 variants/Mb, at least 185 variants/Mb, at least 190 variants/Mb, at least 200 variants/Mb, or at least any other suitable threshold. Additionally, or alternatively, when WES is used to obtain the sequence reads, the threshold may be at most 150 variants/Mb, at most 160 variants/Mb, at most 165 variants/Mb, at most 170 variants/Mb, at most 175 variants/Mb, at most 180 variants/Mb, at most 185 variants/Mb, at most 190 variants/Mb, at most 200 variants/Mb, or at most any other suitable threshold. It should be appreciated that any of the above-listed upper bounds may be coupled with any of the above-listed lower bounds.

FIG. 1C is a diagram depicting an illustrative technique 190 for generating population-specific germline variants 170, according to some embodiments of the technology described herein. As shown in FIG. 1C, the illustrative techniques 190 includes: (a) obtaining sequence reads from non-tumor samples from members of the one or more populations to which the subject (e.g., subject 102 shown in FIG. 1A) belongs; (b) at act 180, aligning the sequence reads to the population-specific genomic reference graph 152; (c) at act 182, using the aligned sequence reads to identify germline variants for each of at least some (e.g., all) of the members of the population(s); and (d) at act 186, merging the identified germline variants to obtain the population-specific germline variants 170.

In some embodiments, technique 190 includes obtaining non-tumor samples from one or more members of one or more population(s) to which the subject (e.g., subject 102 shown in FIG. 1A) belongs. For example, as shown in FIG. 1C, non-tumor sample 174-1 may be obtained from member 172-1, non-tumor sample 174-2 may be obtained from member 172-2, and non-tumor sample 174-3 may be obtained from member 172-3. Though, it should be appreciated that any suitable number of non-tumor samples may be obtained from any particular member and that the members may include any suitable number of members, as aspects of the technology described herein are not limited in this respect. In some embodiments, the non-tumor samples were previously obtained from the members of the one or more population(s). The origin, type, or preparation methods of the non-tumor samples may include any of the embodiments described in the section “Biological Samples.”

In some embodiments, sequence reads are obtained from the non-tumor samples. For example, as shown in FIG. 1C, sequence reads 176-1 are obtained from non-tumor sample 174-1, sequence reads 176-2 are obtained from non-tumor sample 174-2, and sequence reads 176-3 are obtained from non-tumor sample 174-3. In some embodiments, the sequence reads are obtained using at least some of the sequencing techniques described herein, including at least with respect to FIG. 1A, for obtaining sequence reads (e.g., sequence reads 106 shown in FIG. 1A) from a tumor sample (e.g., tumor sample 104). In some embodiments, the sequence reads are obtained from a public database such as, for example, the Sequence Read Archive.

In some embodiments, the sequence reads may be aligned to the population-specific genomic reference graph 152 using any suitable graph alignment techniques, as aspects of the technology described herein are not limited in this respect. In some embodiments, the graph alignment may be performed using dynamic programming. In some embodiments, the graph alignment technique may include a linear alignment technique that has been modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS.”

In some embodiments, results of the alignment, at act 180, include aligned sequence reads for one or more of the members of the population(s). For example, the results of the alignment may include aligned sequence reads for the first member 172-1, aligned sequence reads for the second member 172-2, and aligned sequence reads for the third member 172-3. The aligned sequence reads may include at least some (e.g., all) of the sequence reads 106. In some embodiments, the aligned sequence reads may be associated with information about the alignment. For example, the aligned sequence reads may be associated with at least one position on the population-specific genomic reference graph to which the sequence read aligned. Additionally, or alternatively, the aligned sequence reads may be associated with any other suitable information related to the alignment of the sequence reads at act 180, as aspects of the technology described herein are not limited in this respect.

In some embodiments, identifying germline variants, at act 182, includes identifying where the aligned sequence reads for that individual differs from the genomic reference. In some embodiments, this is performed using variant calling software. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect. GATK software is described by Van der Auwera G A & O'Connor B D. (“Genomics in the Cloud: Using Docker, GATK, and WDL in Terra (1st Edition)”. O'Reilly Media. (2020)), which is incorporated by reference herein in its entirety. SAMtools software is described by Li, H., et al. (“The sequence alignment/map format and SAMtools.” Bioinformatics 25.16 (2009): 2078-2079.), which is incorporated by reference herein in its entirety. BCFtools is described by Li H. (“A statistical framework for SNP calling, mutation discovery, association mapping and population genetical parameter estimation from sequencing data.” Bioinformatics (2011) 27 (21) 2987-93), which is incorporated by reference herein in its entirety.

In some embodiments, germline variants are identified for at least some (e.g., all) of the members of the population(s), at act 182. For example, germline variants 184-1 may be identified for member 172-1, germline variants 184-2 may be identified for member 172-2, and germline variants 184-3 may be identified for member 172-3.

In some embodiments, the germline variants identified for the individual members (e.g., germline variants 184-1, germline variants 184-2, and germline variants 184-3) are merged, at act 186, to obtain the population-specific germline variants 170. In some embodiments, merging the variants includes merging multiple Variant Call Format (VCF) files to generate a single, merged VCF file. The variants may be merged using one or more software tools such as, for example, the “BCFtools merge” software tool. BCFtools is described by Li H. (“A statistical framework for SNP calling, mutation discovery, association mapping and population genetical parameter estimation from sequencing data.” Bioinformatics (2011) 27(21) 2987-93), which is incorporated by reference herein in its entirety.

FIG. 2 is a block diagram of an example system 200 for determining the TMB of a tumor sample from a subject, according to some embodiments of the technology described herein. System 200 includes computing device(s) 210 configured to have software 250 execute thereon to perform various functions in connection with determining TMB for a tumor sample for a subject. In some embodiments, software 250 includes a plurality of modules. A module may include processor-executable instructions that, when executed by at least one computer hardware processor, cause the at least one computer hardware processor to perform the function(s) of the module. Such modules are sometimes referred to herein as “software modules,” each of which includes processor executable instructions configured to perform one or more processes, such as process 300 described herein including at least with respect to FIG. 3.

The computing device(s) 210 may be operated by one or more user(s) 290. For example, the user(s) 290 may include one or more individuals who are treating and/or studying (e.g., doctors, clinicians, researchers, etc.) the subject. Additionally, or alternatively, user(s) 290 may include the subject. In some embodiments, the user(s) 290 may provide, as input to the computing device(s) 210 (e.g., by uploading one or more filed, by interacting with a user interface of the computing device(s) 210, etc.) sequence reads obtained for a tumor sample (e.g., previously obtained for a tumor sample). Additionally, or alternatively, the user(s) 290 may provide input specifying processing or other methods to be performed on the sequence reads. Additionally, or alternatively, the user(s) 290 may access results of processing the sequence reads. For example, the user(s) 290 may access results of determining TMB of the tumor sample.

As shown in FIG. 2, software 250 includes multiple software modules for determining TMB of a tumor sample. Such software modules include a sequence alignment module 252, variant identification module 254, TMB determination module 256, graph generation module 260, population-specific germline variant generation module 264, and therapy recommendation module 262.

In some embodiments, the sequence alignment module 252 obtains sequence reads (e.g., sequence reads 106 shown in FIG. 1A and FIG. 1B) from sequencing platform 270, the user(s) 290 (e.g., by the user(s) uploading the sequence reads), and/or the genomic data store 280. In some embodiments, the sequence alignment module 252 obtains one or more genomic references from user(s) 290 (e.g., by the user(s) 290 uploading the genomic reference, from the graph generation module 260, and/or from the genomic data store 280.

In some embodiments, the sequence alignment module 252 is configured to align the sequence to a population-specific genomic reference graph. For example, the population-specific genomic reference graph may represent a linear reference sequence and population-specific variants relative to the linear reference sequence.

In some embodiments, the sequence alignment module 252 is configured to perform an alignment algorithm to align the sequence reads to the population-specific genomic reference graph. The alignment algorithm may include any suitable alignment algorithm for aligning sequence reads to a genomic reference graph, as aspects of the technology described herein are not limited in this respect. Nonlimiting examples of graph alignment algorithms include, but are not limited to, the alignment algorithms described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”.

In some embodiments, the variant identification module 254 obtains sequence alignment results from sequence alignment module 252, genomic data store 280, and/or user(s) 290 (e.g., by uploading the sequence alignment results). The sequence alignment results may identify one or more positions of a genomic reference to which sequence reads (e.g., the sequence reads from the tumor sample) align. In some embodiments, the variant identification module 254 obtains population-specific germline variants from the population-specific germline variant generation module 264, genomic data store 280, and/or user(s) 290 (e.g., by uploading the sequence alignment results).

In some embodiments, the variant identification module 254 is configured to identify somatic variants based on the sequence alignment results and population-specific germline variants. In some embodiments, identifying the somatic variants includes identifying where at least some of the aligned sequence reads differ from the population-specific genomic reference. In some embodiments, the variant identification module 254 uses variant calling software to identify somatic variants based on alignment results. Nonlimiting examples of variant calling software include Mutect2 software, rasm software, Strelka2 software, VarScan2 software, or any other suitable somatic variant calling software as aspects of the technology described herein are not limited in this respect.

In some embodiments, the population-specific germline variant generation module 264 obtains sequence reads (e.g., sequence reads 176-1, 176-2, and 176-3 shown in FIG. 1C) from sequencing platform 270, the user(s) 290 (e.g., by the user(s) uploading the sequence reads), and/or the genomic data store 280. In some embodiments, the population-specific germline variant generation module 264 obtains one or more genomic references from user(s) 290 (e.g., by the user(s) 290 uploading the genomic reference), from the graph generation module 260, and/or from the genomic data store 280.

In some embodiments, the population-specific germline variant generation module 264 is configured to generate population-specific germline variants. For example, the population-specific germline variants may be used by the variant identification module 254 as part of identifying somatic variants. In some embodiments, the population-specific germline variant generation module 264 is configured to generate population-specific germline variants using any suitable technique, as aspects of the technology described herein are not limited in this respect. For example, the population-specific germline variant generation module 264 may be configured to implement technique 190 described herein including at least with respect to FIG. 1C. For example, the population-specific germline variant generation module 264 may be configured to: (a) align sequence reads from member(s) of one or more populations to a genomic reference (e.g., a population-specific genomic reference) to obtain; (b) identify germline variants for each member based on results of the aligning; and (c) merge the germline variants to obtain the population-specific germline variants.

In some embodiments, the graph generation module 260 obtains one or more genomic references (e.g., a linear genomic reference) from the genomic data store 280 and/or user(s) 290 (e.g., by user(s) uploading the genomic reference(s)). In some embodiments, the graph generation module 260 obtains variants from genomic data store 280 and/or user(s) 290 (e.g., by the user(s) uploading the variants).

In some embodiments, the graph generation module 260 is configured to generate one or more genomic reference graphs. In some embodiments, generating a genomic reference graph includes augmenting a linear genomic reference with one or more variants (e.g., common among the global population, common among specific population(s) and/or identified for specific individuals). In some embodiments, this may be achieved by generating one or more data structures having node elements and edge elements that represent the linear genomic reference, and augmenting the data structure with node elements and edge elements that represent variants of the linear genomic reference. A node element may be represented as an object, and an object may store a pointer that represents an edge. Example techniques for generating a genomic reference graph are described by Rakocevic, G., et al. (“Fast and accurate genomic analysis using genome graphs.” Nat Genet. 51.2 (2019): 354-362), which is incorporated by reference herein in its entirety.

In some embodiments, the graph generation module 260 is configured to generate a population-specific genomic reference graph. For example, in some embodiments, the graph generation module 260 may generate a genomic reference graph that represents a linear genomic reference and variants that are common to one or more specific populations.

For example, the specific populations may include those to which the subject belongs. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384), which is incorporated by reference herein in its entirety.

In some embodiments, the TMB determination module 256 obtains variants from variant identification module 254, user(s) 290 (e.g., by uploading variants), and/or genomic data store 280.

In some embodiments, the TMB determination module 256 is configured to determine the TMB of a tumor sample. In some embodiments, the TMB determination module 256 is configured to determine TMB using the somatic variants identified by variant identification module 254. Additionally, alternatively, the TMB determination module 256 may be configured to determine the TMB using information about the sequencing of the tumor sample. For example, the information about the sequencing of the tumor sample may include an indication of the type of sequencing using (e.g., WGS or WES), a size of the genomic region sequenced (e.g., number of base pairs), or any other suitable sequencing information. In some embodiments, the TMB determination module 256 is configured to determine the TMB at least in part be determining a ratio between the number of somatic variants identified to the size of the region of the genome of the tumor sample that was sequenced. As one nonlimiting example, TMB may be determined by identifying the total number of somatic variants per megabase. In some embodiments, in determining the TMB, the TMB determination module 256 is configured to implement one or more of the techniques described by Büttner, R., et al., (“Implementing TMB measurements in clinical practice: considerations on assay requirements.” ESMO open 4.1 (2019): e000442.).

In some embodiments, the therapy recommendation module 262 may obtain the TMB from the TMB determination module 256, the genomic data store 280, and/or user(s) 290 (e.g., by the user(s) uploading the TMB). Additionally, or alternatively, the therapy recommendation module 262 may obtain information about one or more therapies from the genomic data store 280 and/or user(s) 290 (e.g., by the user(s) uploading the information about the one or more therapies). For example, therapy information may indicate one or more therapies, data indicative of the response of subject(s) to one or more therapies, or any other suitable information.

In some embodiments, the therapy recommendation module 262 is configured to determine a therapy recommendation for the subject. For example, the therapy recommendation module 262 may be configured to identify one or more therapies to be administered to the subject. In some embodiments, this may include predicting a response of the subject to one or more therapies based on the TMB determined for a tumor sample from the subject. For example, if the TMB is greater than or equal to a threshold, the techniques may include determining that the subject will respond positively to administration of an immunotherapy. Example thresholds are described herein including at least with respect to FIG. 1B. Example therapies are described herein including in the section “Therapies.”

In some embodiments, software 250 further includes user interface module 258. User interface module 258 may be configured to generate a graphical user interface (GUI) through which the user may provide input and view information generated by software 250. For example, in some embodiments, the user interface module 258 may be a webpage or web application accessible through an Internet browser. In some embodiments, the user interface module 258 may generate a graphical user interface (GUI) of an app executing on the user's mobile device. In some embodiments, the user interface module 258 may generate a GUI on a sequencing platform, such as sequencing platform 270. In some embodiments, the user interface module 258 may generate a number of selectable elements through which a user may interact. For example, the user interface module 258 may generate dropdown lists, checkboxes, text fields, or any other suitable element.

In some embodiments, the user interface module 258 is configured to generate a GUI including one or more results of processing sequence reads obtained from the tumor sample from the subject. For example, the GUI may include an indication of the TMB determined for the tumor sample. Additionally, or alternatively, in some embodiments, the GUI may include an indication of one or more therapies recommended for treating the subject. It should be appreciated that the GUI may include any other suitable information, displayed in any suitable manner, as aspects of the technology described herein are not limited in this respect.

As shown in FIG. 2, system 200 also includes sequencing platform 270. In some embodiments, sequence reads are obtained from the sequencing platform 270. For example, the sequence alignment module 252 may obtain (either pull or be provided) the sequence reads from the sequencing platform 270. The sequencing platform 270 may be one of any suitable type such as, for example, any of the sequencing platforms described herein including at least with respect to FIG. 1A and with respect to the section “Sequencing Data.”

System 200 further includes genomic data store 280. In some embodiments, the genomic data store 280 stores sequence reads that were previously obtained for one or more subjects (e.g., using sequencing platform 270). Additionally, or alternatively, genomic data store 280 stores one or more genomic references (e.g., linear genomic references and/or genomic reference graph(s)). Additionally, or alternatively, genomic data store 280 stores sequence alignment results (e.g., obtained from sequence alignment module 252) and/or variant identification results (e.g., obtained from variant identification module 254). Additionally, or alternatively, genomic data store 280 may store information about therapies associated with TMB values. It should be appreciated that the genomic data store 280 may store any other suitable type of information, as aspects of the technology described herein are not limited in this respect.

The genomic data store 280 may be of any suitable type (e.g., database system, multi-file, flat file, etc.) and may store genomic data in any suitable way in any suitable format, as aspects of the technology described herein are not limited in this respect. The genomic data store 280 may be part of or external to the computing device(s) 210.

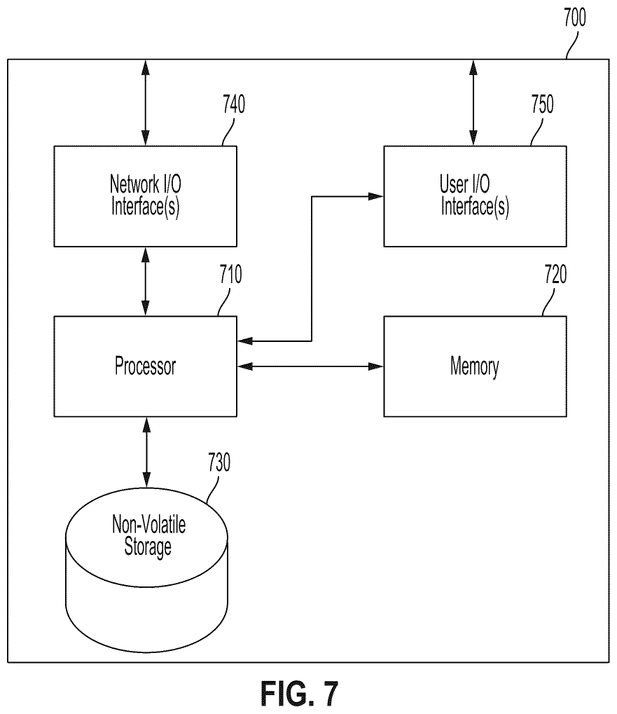

FIG. 3 is a flowchart of an illustrative process 300 for determining the TMB of a tumor sample from a subject, according to some embodiments of the technology described herein. One or more acts (e.g., all acts) of process 300 may be performed automatically by any suitable computing device(s). For example, the act(s) may be performed by a laptop computer, a desktop computer, one or more servers, in a cloud computing environment, computing device 700 as described herein including with respect to FIG. 7, and/or in any other suitable way.

At act 302, sequence reads are obtained for the subject. In some embodiments, the sequence reads had been previously obtained by sequencing a tumor sample from a subject. In some embodiments, the tumor sample includes cells from a benign tumor, e.g., non-cancerous cells. In some embodiments, the tumor sample includes cells from a premalignant tumor, e.g., precancerous cells. In some embodiments, the tumor sample includes cells from a malignant tumor, e.g., cancerous cells. Examples of tumors include, but are not limited to, adenomas, fibromas, hemangiomas, lipomas, cervical dysplasia, metaplasia of the lung, leukoplakia, carcinoma, sarcoma, germ cell tumors, sex cord-stromal tumors, neuroendocrine tumors, gastrointestinal stromal tumors, and blastoma. Examples of tumor samples are described herein including at least with respect to FIG. 1A and with respect to the section “Biological Samples.”

In some embodiments, the sequence reads were previously obtained using a sequencing platform such as a next-generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequence reads may be the result of non-next generation sequencing (e.g., Sanger sequencing). Examples of sequencing techniques are described herein including at least with respect to the section “Sequencing Data.”

In some embodiments, the sequence reads are obtained, at act 302, from a sequencing platform (e.g., sequencing platform 270 shown in FIG. 2), a data store (e.g., genomic data store 280 show in FIG. 2), from one or more user(s) of the computing device used to implement process 300 (e.g., by uploading the sequence reads), or from any other suitable source, as aspects of the technology described herein are not limited in this respect.

In some embodiments, the obtained sequence reads include any suitable number of sequence reads such as, for example, a number of sequence reads between 1,000 and 100,000,000 sequence reads, between 10,000 and 10,000,000 sequence reads, between 100,000 and 1,000,000 sequence reads, or any other suitable number of sequence reads, as aspects of the technology described herein are not limited in this respect. In some embodiments, the obtained sequence reads may include at least 1,000, at least 10,000, at least 100,000, at least 1,000,000, at least 10,000,000, at least 100,000,000, or at least any other suitable number of sequence reads. In some embodiments, the obtained sequence reads may include at most 1,000, at most, 10,000, at most 100,000, at most 1,000,000, at most 10,000,000, at most 100,000,000, or at most any other suitable number of sequence reads. It should be appreciated that any of the above-listed upper bounds may be coupled with any of the above-listed lower bounds.

In some embodiments, the sequence reads obtained at act 302 are in any suitable format. For example, the sequence reads may be specified in one or more files such as FASTQ files.

At act 304, the sequence reads are aligned to a population-specific genomic reference by using at least one data structure representing the population-specific genomic reference graph. The population-specific genomic reference may represent a linear reference sequence and population-specific variants relative to the linear reference sequence. The linear reference sequence may include a human genome reference sequence such as, for example, human genome version 19 (hg19), hg38, Genome Reference Consortium human reference 38 (GRCh38), GRCh37, or any other suitable human genome reference sequence, as aspects of the technology described herein are not limited in this respect. The population-specific variants may represent variants that are common among members of one or more populations to which the subject belongs. Nonlimiting examples of populations include African ancestry (AFR), American ancestry (AMR), South-Asian ancestry (SAS), Eastern-Asian ancestry (EAS), and European ancestry (EUR). Variants that are specific to particular populations may be obtained from any suitable source such as, for example, the 1000 Genomes Project consortium. The population(s) to which the subject belongs may be identified using any suitable techniques, as aspects of the technology are not limited in this respect. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384), which is incorporated by reference herein in its entirety.

In some embodiments, the data structure representing the population-specific genomic reference graph specifies nodes and edges of the population-specific genomic reference graph. The nodes may represent nucleotide sequences stored as respective strings of one or more symbols, and each of the edges may represent a connection between at least two of the nodes. Alternatively, the edges may represent nucleotide sequences stored as respective strings of one or more symbols, and each of the nodes may represent a connection between at least two of the edges. In some embodiments, the data structure includes objects that represent the nodes and pointers that represent the edges. The data structure may be stored in at least one non-transitory computer-readable storage medium. As one non-limiting example, the data structure may be a directed acyclic graph (DAG). Example techniques for generating a genomic reference graph are described by Rakocevic, G., et al. (“Fast and accurate genomic analysis using genome graphs.” Nat Genet. 51.2 (2019): 354-362).

In some embodiments, the sequence reads are aligned to the population-specific genomic reference graph, at act 304, using any suitable graph alignment techniques, as aspects of the technology described herein are not limited in this respect. In some embodiments, the graph alignment may be performed using dynamic programming. In some embodiments, one or more linear sequence alignment techniques may be modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”.

In some embodiments, one or more files are output as a result of aligning the sequence reads to the population-specific genomic reference graph. The file(s) may include information representing the aligned sequence reads with respect to the population-specific genomic reference graph. The file(s) may be in any suitable format for representing aligned sequences such as, for example, sequence alignment map (SAM) file format or binary alignment map (BAM) file format, or compressed reference-oriented alignment map (CRAM) file format.

At act 306, a plurality of somatic variants is identified based on results of aligning the sequence reads to the population-specific genomic reference graph. In some embodiments, the variant identification is performed using somatic variant calling software. Nonlimiting examples of somatic variant calling software include Mutect2 software, rasm software, Strelka2 software, VarScan2 software, or any other suitable somatic variant calling software as aspects of the technology described herein are not limited in this respect. Mutect2 software is described by Cibulskis, K., et al. (“Sensitive detection of somatic point mutations in impure and heterogeneous cancer samples.” Nature biotechnology 31.3 (2013): 213-219), which is incorporated by reference herein in its entirety. Strelka2 software is described by Saunders, C., et al. (“Strelka: accurate somatic small-variant calling from sequenced tumor-normal sample pairs.” Bioinformatics 28.14 (2012): 1811-1817), which is incorporated by reference herein in its entirety. VarScan2 software is described by Koboldt, D., et al. (“VarScan 2: somatic and copy number alteration discovery in cancer by exome sequencing.” Genome research 22.3 (2012): 568-576), which is incorporated by reference herein it its entirety.

In some embodiments, the somatic variants identified, at act 306, include non-synonymous variants. Non-synonymous variants are variants that lead to a change in the amino acid sequence of a protein.

In some embodiments, population-specific germline variants may be used to identify the somatic variants at act 306. The population-specific germline variants may include germline variants that have been identified for one or more non-tumor samples (e.g., biological samples that are believed to have less than a threshold number of somatic variants). In some embodiments, the non-tumor samples are obtained, or were previously obtained, from members of the same one or more populations to which the subject (e.g., subject 102 shown in FIG. 1A) belongs. The population-specific germline variants may be used (e.g., used by somatic variant calling software) to distinguish between somatic variants and germline variants that are common among the members of the population(s), thereby resulting in a more accurate estimation of somatic variants for the subject. For example, if a variant is found is the population-specific germline variants, it may be filtered out and excluded from the final estimation of somatic variants.

The population-specific germline variants may be obtained using any suitable techniques, as aspects of the technology described herein are not limited in this respect. In some embodiments, the population-specific germline variants may be obtained using the population-specific genomic reference graph used for aligning the sequence reads at act 304. For example, the population-specific germline variants may be obtained using the illustrative techniques 190 described herein with respect to FIG. 1C. In some embodiments, the population-specific germline variants are obtained from a public database such as, for example, the public Genome Analysis Toolkit (GATK) population-specific germline variants.

In some embodiments, the population-specific germline variants are specified in one or more files of any suitable format. For example, the population-specific germline variants may be specified in one or more Variant Call Format (VCF) files.

In some embodiments, the output of act 306 includes one or more files that include information indicative of the somatic variants identified for the subject. The file(s) may be in any suitable format such as, for example, VCF.

At act 308, TMB of the tumor sample is determined using the identified plurality of somatic variants. In some embodiments, determining the TMB of the tumor sample includes determining a ratio between the number of somatic variants identified at act 306 and the size of the genomic region of the tumor sample that was sequenced. The size of the genomic region may be measured in any suitable unit of measurement, as aspects of the technology described herein are not limited in this respect. For example, the TMB may be determined by identifying the total number of somatic variants per megabase. Techniques and consideration for determining TMB are described by Büttner, R., et al., (“Implementing TMB measurements in clinical practice: considerations on assay requirements.” ESMO open 4.1 (2019): e000442.).

At act 310, the determined TMB is used to determine to administer an immunotherapy to the subject. The immunotherapy may include any suitable therapy such as, for example, an immune checkpoint inhibitor or any of the immunotherapies described in the section “Therapies.”

In some embodiments, determining to administer the therapy to the subject includes determining that the TMB is greater than or equal to a threshold TMB. In some embodiments, the threshold depends on the type of sequencing used to obtain the sequence reads. For example, when WGS is used to obtain the sequence reads, the threshold may be between 8 variants/Mb and 12 variants/Mb, between 9 variants/Mb and 11 variants/Mb, or any other suitable threshold. Additionally, or alternatively, when WGS is used to obtain the sequence reads, the threshold may be at least 8 variants/Mb, at least 9 variants/Mb, at least 10 variants/Mb, at least 11 variants/Mb, at least 12 variants/Mb, or at least any other suitable threshold. Additionally, or alternatively, when WGS is used to obtain the sequence reads, the threshold may be at most 8 variants/Mb, at most 9 variants/Mb, at most 10 variants/Mb, at most 11 variants/Mb, at most 12 variants/Mb, or at most any other suitable threshold. It should be appreciated that any of the above-listed upper bounds may be coupled with any of the above-lister lower bounds. In some embodiments, when WES is used to obtain the sequence reads, the threshold may be between 150 variants/Mb and 200 variants/Mb, between 160 and 190 variants/Mb, between 165 variants/Mb and 185 variants/Mb, between 170 variants/Mb and 180 variants/Mb, or any other suitable threshold. Additionally, or alternatively, when WES is used to obtain the sequence reads, the threshold may be at least 150 variants/Mb, at least 160 variants/Mb, at least 165 variants/Mb, at least 170 variants/Mb, at least 175 variants/Mb, at least 180 variants/Mb, at least 185 variants/Mb, at least 190 variants/Mb, at least 200 variants/Mb, or at least any other suitable threshold. Additionally, or alternatively, when WES is used to obtain the sequence reads, the threshold may be at most 150 variants/Mb, at most 160 variants/Mb, at most 165 variants/Mb, at most 170 variants/Mb, at most 175 variants/Mb, at most 180 variants/Mb, at most 185 variants/Mb, at most 190 variants/Mb, at most 200 variants/Mb, or at most any other suitable threshold. It should be appreciated that any of the above-listed upper bounds may be coupled with any of the above-lister lower bounds. In some embodiments, when the sequence reads are obtained using a type of sequencing other than WES or WGS, any suitable threshold may be used, as aspects of the technology described herein are not limited in this respect.

The immunotherapy may include any suitable therapy such as, for example, an immune checkpoint inhibitor or any of the immunotherapies described in the section “Therapies.”

At act 312, the immunotherapy is administered to the subject. The immunotherapy may be administered using any suitable techniques, as aspects of the technology described herein are not limited in this respect. For example, the therapy may be administered according to any of the embodiments described in the section “Therapies.”

It should be appreciated that process 300 may include one or more additional or alternative acts not shown in FIG. 3. For example, process 300 may exclude one or both of acts 308 and 310.

### EXAMPLE 1

### Genetic Variation Among Members of a Population

This example shows that tumor samples obtained from members of a population share common genetic variants.

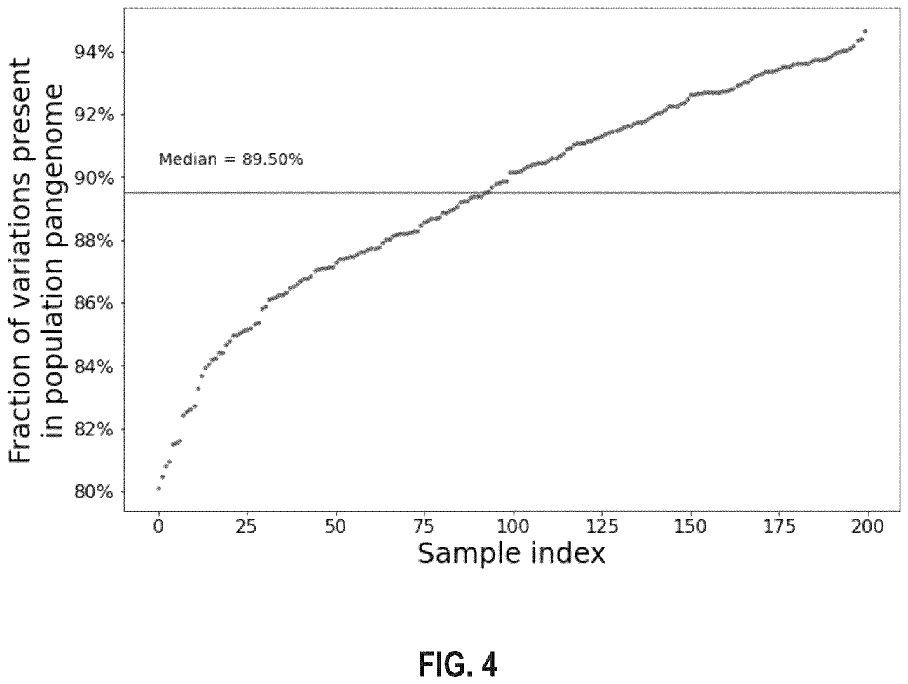

An experiment was performed to determine the fraction of individual variation shared with a population-specific genomic reference. In the experiment, sequence reads obtained from tumor samples from 200 Brazilian individuals were compared to a population-specific genomic reference graph that represented variants common to the Brazilian population. As shown in FIG. 4, each sample shared over 80% of individual genetic variation with the population-specific genomic reference, with a median of 89.50% over all samples. This suggests that the population-specific genomic reference is effective in representing germline variants that are common among members of one or more populations.

### EXAMPLE 2

### Improvement in Accuracy and Precision

These examples show that the techniques developed by the inventors for determining TMB of a tumor sample are an improvement over conventional techniques for determining TMB using sequence reads. In the examples, the techniques developed by the inventors will be referred to as “GRAF.”

Experiments were performed to benchmark the performance of the GRAF techniques. The performance of the GRAF techniques was compared with that of the Genome Analysis Toolkit-Broad Institute Best Practice Somatic Analysis Pipeline (GATK-BROAD). The techniques were compared in two scenarios: a tumor-only scenario and a tumor-normal scenario.

In the tumor-only scenario, only tumor samples were provided as input without any corresponding non-tumor (“normal”) samples. In this scenario, the Breast Cancer Benchmark Sample was employed. This sample has been validated and published The Somatic Mutation Working Group of the Sequencing Quality Control Phase II Consortium (“Establishing community reference samples, data and call sets for benchmarking cancer mutation detection using whole-genome sequencing.” Nat. Biotechnol. 39, 1151-1160 (2021)), which is incorporated by reference herein in its entirety. The Breast Cancer Benchmark Sample is recognized as a reference cancer sample in somatic analyses. The evaluation considered both the designated benchmark region (high confidence region) specified for the truth set, as well as the whole exome sequencing region.

In the tumor-normal scenario, tumor samples and corresponding non-tumor samples were provided as input. These samples were previously reported by Butler, T. et al., (“Exome Sequencing of Cell-Free DNA from Metastatic Cancer Patients Identifies Clinically Actionable Mutations Distinct from Primary Disease.” PLOS One 10, e0136407 (2015)), which is incorporated by reference herein in its entirety.

The results indicate that the GRAF techniques enhance the precision of somatic calling, with a pronounced improvement observed in the tumor-only scenario. As shown in FIG. 5, with respect to the tumor-only analysis, the GRAF techniques yielded a TMB value that is more precisely aligned with the “true” TMB score for the benchmark sample, where the somatic variants have been validated inside the high confidence region covering a substantial portion of the genome. As shown in FIG. 6, with respect to the tumor-only analysis, the GRAF techniques led to more precise TMB scores across three unrelated samples extracted from the biopsies of different cancer types.

### Computer Implementation

An illustrative implementation of a computer system 700 that may be used in connection with any of the embodiments of the technology described herein (e.g., such as the process of FIG. 3) is shown in FIG. 7. The computer system 700 includes one or more processors 710 and one or more articles of manufacture that comprise non-transitory computer-readable storage media (e.g., memory 720 and one or more non-volatile storage media 730). The processor 710 may control writing data to and reading data from the memory 720 and the non-volatile storage device 730 in any suitable manner, as the aspects of the technology described herein are not limited to any particular techniques for writing or reading data. To perform any of the functionality described herein, the processor 710 may execute one or more processor-executable instructions stored in one or more non-transitory computer-readable storage media (e.g., the memory 720), which may serve as non-transitory computer-readable storage media storing processor-executable instructions for execution by the processor 710.

Computing device 700 may include a network input/output (I/O) interface 740 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 700 may also include one or more user I/O interfaces 750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### Biological Samples

Any of the methods, systems, or other claimed elements may use or be used to analyze a biological sample from a subject. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, sex cord-stromal tumors, neuroendocrine tumors, gastrointestinal stromal tumors, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of a tissue, in some embodiments, refers to a sample comprising cells from a tissue. In some embodiments, the sample of the tumor comprises non-cancerous cells from a tissue. In some embodiments, the sample of the tumor comprises precancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, breast, and/or any type of tissue (e.g., muscle tissue, epithelial tissue, connective tissue, or nervous tissue).

Any of the biological samples described herein may be obtained from the subject using any known technique. See, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February;21 (2): 253-5), and Biological sample collection, processing, storage and information management by Vaught and Henderson (IARC Sci Publ. 2011; (163): 23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixative. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextronse (e.g., for blood specimens). In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. As a non-limiting example, the second or subsequent biological sample may be useful in determining whether the cancer in each biological sample has different characteristics (e.g., in the case of biological samples taken from two physically separate tumors in a subject) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

In some embodiments, a sample from which RNA and/or DNA is extracted (e.g., a sample of tumor, or a blood sample) is sufficiently large such that at least 2 μg (e.g., at least 2 μg, at least 2.5 μg, at least 3 μg, at least 3.5 μg or more) of DNA can be extracted from it. In some embodiments, the sample from which RNA and/or DNA is extracted can be peripheral blood mononuclear cells (PBMCs). In some embodiments, the sample from which RNA and/or DNA is extracted can be any type of cell suspension. In some embodiments, a sample from which RNA and/or DNA is extracted (e.g., a sample of tumor, or a blood sample) is sufficiently large such that at least 1.8 μg DNA can be extracted from it. In some embodiments, at least 50 mg (e.g., at least 1 mg, at least 2 mg, at least 3 mg, at least 4 mg, at least 5 mg, at least 10 mg, at least 12 mg, at least 15 mg, at least 18 mg, at least 20 mg, at least 22 mg, at least 25 mg, at least 30 mg, at least 35 mg, at least 40 mg, at least 45 mg, or at least 50 mg) of tissue sample is collected from which RNA and/or DNA is extracted. In some embodiments, at least 20 mg of tissue sample is collected from which RNA and/or DNA is extracted. In some embodiments, at least 30 mg of tissue sample is collected. In some embodiments, at least 10-50 mg (e.g., 10-50 mg, 10-15 mg, 10-30 mg, 10-40 mg, 20-30 mg, 20-40 mg, 20-50 mg, or 30-50 mg) of tissue sample is collected from which RNA and/or DNA is extracted. In some embodiments, at least 30 mg of tissue sample is collected. In some embodiments, at least 20-30 mg of tissue sample is collected from which RNA and/or DNA is extracted. In some embodiments, a sample from which RNA and/or DNA is extracted (e.g., a sample of tumor, or a blood sample) is sufficiently large such that at least 0.2 μg (e.g., at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of DNA can be extracted from it. In some embodiments, a sample from which RNA and/or DNA is extracted (e.g., a sample of tumor, or a blood sample) is sufficiently large such that at least 0.1 μg (e.g., at least 100 ng, at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of DNA can be extracted from it.

### Subjects

Aspects of this disclosure relate to a tumor sample that has been obtained from one or more subjects. In some embodiments, a subject is a mammal (e.g., a human, a mouse, a cat, a dog, a horse, a hamster, a cow, a pig, or other domesticated animal, a farm animal (e.g., livestock), a sport animal, a laboratory animal, a pet, and a primate). In some embodiments, a subject is a human. In some embodiments, a subject is an adult human (e.g., of 18 years of age or older). In some embodiments, a subject is a child (e.g., less than 18 years of age).

### Sequencing Data

Aspects of the disclosure may be implemented using sequencing data. For example, aspects of the disclosure relate to methods for determining TMB of a tumor sample analyzing sequencing data, such as sequence reads, from the tumor sample.

In some embodiments, sequencing data may be generated using a nucleic acid from a sample from a subject. In some embodiments, the sequencing data may indicate a nucleotide sequence of DNA from a previously obtained tumor sample of a subject having, suspected of having, or at risk of having a disease. In some embodiments, the nucleic acid is deoxyribonucleic acid (DNA). In some embodiments, the nucleic acid is prepared such that the whole genome is present in the nucleic acid. When nucleic acids are prepared such that the whole genome is sequenced, it is referred to as whole genome sequencing (WGS). In some embodiment, the nucleic acid is prepared such that fragmented DNA is present in the nucleic acid. In some embodiments, the nucleic acid is processed such that only the protein coding regions of the genome remain (e.g., exomes). When nucleic acids are prepared such that only the exomes are sequenced, it is referred to as whole exome sequencing (WES). A variety of methods are known in the art to isolate the exomes for sequencing, for example, solution-based isolation wherein tagged probes are used to hybridize the targeted regions (e.g., exomes) which can then be further separated from the other regions (e.g., unbound oligonucleotides). These tagged fragments can then be prepared and sequenced.

In some embodiments, the sequencing data may include DNA sequencing data, DNA exome sequencing data (e.g., from whole exome sequencing (WES)), DNA genome sequencing data (e.g., from whole genome sequencing (WGS), shallow whole genome sequencing (sWGS), etc.), gene sequencing data, bias-corrected gene sequencing data, or any other suitable type of sequencing data comprising data obtained from a sequencing platform and/or comprising data derived from data obtained from a sequencing platform.

DNA sequencing data, in some embodiments, may include a level of DNA (e.g., copy number of a chromosome, gene, or other genomic region) in a sample from a subject. The level of DNA in a sample from a subject having cancer may be elevated compared to the level of DNA in a sample from a subject not having cancer, e.g., a gene duplication in a cancer patient's subject's sample. The level of DNA in a sample from a subject having cancer may be reduced compared to the level of DNA in a sample from a subject not having cancer, e.g., a gene deletion in a cancer patient's subject's sample.

DNA sequencing data, in some embodiments, includes DNA sequence reads and/or information derived from DNA sequence reads. A DNA sequence read refers to an inferred sequence of base pairs corresponding to all or part of a DNA fragment.

DNA sequencing data, in some embodiments, includes data obtained by processing a tumor sample (e.g., DNA (e.g., coding or non-coding genomic DNA) present in a tumor sample) using a sequencing apparatus. DNA that is present in a sample may or may not be transcribed, but it may be sequenced using DNA sequencing platforms. Such data may be useful, in some embodiments, to determine whether the patient subject has one or more variants associated with a particular cancer.

Sequencing data may include data generated by the nucleic acid sequencing protocol (e.g., the series of nucleotides in a nucleic acid molecule identified by any suitable generation of sequencing (Sanger sequencing, Illumina®, next-generation sequencing (NGS) etc.), as well as information contained therein (e.g., information indicative of source, tissue type, etc.) which may also be considered information that can be inferred or determined from the sequencing data.

DNA sequencing data may be acquired using any method known in the art including any known method of DNA sequencing. For example, DNA sequencing may be used to identify one or more variants in the DNA of a subject. Any technique used in the art to sequence DNA may be used with the methods and compositions described herein. As a set of non-limiting examples, the DNA may be sequenced through single-molecule real-time sequencing, ion torrent sequencing, pyrosequencing, sequencing by synthesis, sequencing by ligation (SOLID sequencing), nanopore sequencing, or Sanger sequencing (chain termination sequencing).

In some embodiments, the sequencing data may be obtained using a sequencing platform such as a next generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequencing data may be the result of non-next generation sequencing (e.g., Sanger sequencing).

In some embodiments, sequencing data comprises more than 5 kilobases (kb). In some embodiments, the size of the obtained sequencing data is at least 10 kb. In some embodiments, the size of the obtained sequencing data is at least 100 kb. In some embodiments, the size of the obtained sequencing data is at least 500 kb. In some embodiments, the size of the obtained sequencing data is at least 1 megabase (Mb). In some embodiments, the size of the obtained sequencing data is at least 10 Mb. In some embodiments, the size of the obtained sequencing data is at least 100 Mb. In some embodiments, the size of the obtained sequencing data is at least 500 Mb. In some embodiments, the size of the obtained sequencing data is at least 1 gigabase (Gb). In some embodiments, the size of the obtained sequencing data is at least 10 Gb. In some embodiments, the size of the obtained sequencing data is at least 100 Gb. In some embodiments, the size of the obtained sequencing data is at least 500 Gb.

### Therapies

Aspects of the disclosure relate to methods of identifying or selecting a therapy agent for a subject based upon a determination of TMB for a tumor sample obtained from a subject. The disclosure is based, in part, on the recognition that subjects having a TMB greater than or equal to a threshold TMB may have an increased likelihood of responding to certain therapies relative to subjects that have a TMB less than the threshold.

In some embodiments, the therapeutic agents are immune checkpoint inhibitors. Examples of immune checkpoint inhibitors include pembrolizumab, ipilimumab, nivolumab, cemiplimab, dostarlimab, atezolizumab, durvalumab, and avelumab.

In some embodiments, methods described by the disclosure further comprise a step of administering one or more therapeutic agents to the subject based upon the determination of the TMB of the tumor sample. In some embodiments, a subject is administered one or more (e.g., 1, 2, 3, 4, 5, or more) immune checkpoint inhibitors.

Aspects of the disclosure relate to methods of treating a subject having (or suspected or at risk of having) cancer based upon a determination of TMB of a tumor sample from the subject. In some embodiments, the methods comprise administering one or more (e.g., 1, 2, 3, 4, 5, or more) therapeutic agents to the subject.

The subject to be treated by the methods described herein may be a human subject having, suspected of having, or at risk for a cancer. Examples of a cancer include, but are not limited to, melanoma, lung cancer, brain cancer, breast cancer, colorectal cancer, pancreatic cancer, liver cancer, skin cancer, kidney cancer, bladder cancer, ovarian cancer, cervical cancer, or prostate cancer. At the time of diagnosis, the cancer may be cancer of unknown primary. The subject to be treated by the methods described herein may be a mammal (e.g., may be a human). Mammals include but are not limited to: a human, a mouse, a cat, a dog, a horse, a hamster, a cow, a pig, or other domesticated animal, a farm animal (e.g., livestock), a sport animal, a laboratory animal, a pet, and a primate.

A subject having a cancer may be identified by routine medical examination, e.g., laboratory tests, biopsy, PET scans, CT scans, or ultrasounds. A subject suspected of having a cancer might show one or more symptoms of the disorder, e.g., unexplained weight loss, fever, fatigue, cough, pain, skin changes, unusual bleeding or discharge, and/or thickening or lumps in parts of the body. A subject at risk for a cancer may be a subject having one or more of the risk factors for that disorder. For example, risk factors associated with cancer include, but are not limited to, (a) viral infection (e.g., herpes virus infection), (b) age, (c) family history, (d) heavy alcohol consumption, (e) obesity, and (f) tobacco use.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer.

Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

When the anti-cancer therapeutic agent is not an antibody, it may be administered at the rate of about 0.1 to 300 mg/kg of the weight of the subject divided into one to three doses, or as disclosed herein. In some embodiments, for an adult subject of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the subject's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

In some embodiments, the anti-cancer therapeutic agent described herein is administered to a subject in need of the treatment at an amount sufficient to reduce cancer (e.g., tumor) growth by at least 10% (e.g., 20%, 30%, 40%, 50%, 60%, 70%, 80%, 90% or greater). In some embodiments, the anti-cancer therapeutic agent described herein is administered to a subject in need of the treatment at an amount sufficient to reduce cancer cell number or tumor size by at least 10% (e.g., 20%, 30%, 40%, 50%, 60%, 70%, 80%, 90% or more). In other embodiments, the anti-cancer therapeutic agent is administered in an amount effective in altering cancer type. Alternatively, the anti-cancer therapeutic agent is administered in an amount effective in reducing tumor formation or metastasis.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (e.g., glycerol, propylene glycol, liquid polyethylene glycol, and the like). For intravenous injection, water soluble anti-cancer therapeutic agents can be administered by the drip method, whereby a pharmaceutical formulation containing the antibody and a physiologically acceptable excipients is infused. Physiologically acceptable excipients may include, for example, 5% dextrose, 0.9% saline, Ringer's solution, and/or other suitable excipients. Intramuscular preparations, e.g., a sterile formulation of a suitable soluble salt form of the anti-cancer therapeutic agent, can be dissolved and administered in a pharmaceutical excipient such as Water-for-Injection, 0.9% saline, and/or 5% glucose solution.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. The contents of each of the foregoing are incorporated by reference herein for this purpose.

Therapeutic compositions containing a polynucleotide may be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

Therapeutic polynucleotides and polypeptides can be delivered using gene delivery vehicles. The gene delivery vehicle can be of viral or non-viral origin (e.g., Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). The contents of each of the foregoing are incorporated by reference herein for this purpose. Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed. The contents of each of the foregoing are incorporated by reference herein for this purpose.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP U.S. Pat. No. 524,968. Additional approaches are described in Philip, Mol. Cell. Biol. (1994) 14:2411, and in Woffendin, Proc. Natl. Acad. Sci. (1994) 91:1581. The contents of each of the foregoing are incorporated by reference herein for this purpose.

It is also apparent that an expression vector can be used to direct expression of any of the protein-based anti-cancer therapeutic agents (e.g., anti-cancer antibody). For example, peptide inhibitors that are capable of blocking (from partial to complete blocking) a cancer-causing biological activity are known in the art.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a subject subjected to the treatment. Alternatively, or in addition to, treatment efficacy can be assessed by monitoring tumor type over the course of treatment (e.g., before, during, and after treatment).

A subject having cancer may be treated using any combination of anti-cancer therapeutic agents or one or more anti-cancer therapeutic agents and one or more additional therapies (e.g., surgery and/or radiotherapy). The term combination therapy, as used herein, embraces administration of more than one treatment (e.g., an antibody and a small molecule or an antibody and radiotherapy) in a sequential manner, that is, wherein each therapeutic agent is administered at a different time, as well as administration of these therapeutic agents, or at least two of the agents or therapies, in a substantially simultaneous manner.

Sequential or substantially simultaneous administration of each agent or therapy can be affected by any appropriate route including, but not limited to, oral routes, intravenous routes, intramuscular, subcutaneous routes, and direct absorption through mucous membrane tissues. The agents or therapies can be administered by the same route or by different routes. For example, a first agent (e.g., a small molecule) can be administered orally, and a second agent (e.g., an antibody) can be administered intravenously.

As used herein, the term “sequential” means, unless otherwise specified, characterized by a regular sequence or order, e.g., if a dosage regimen includes the administration of an antibody and a small molecule, a sequential dosage regimen could include administration of the antibody before, simultaneously, substantially simultaneously, or after administration of the small molecule, but both agents will be administered in a regular sequence or order. The term “separate” means, unless otherwise specified, to keep apart one from the other. The term “simultaneously” means, unless otherwise specified, happening or done at the same time, i.e., the agents are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents or therapies described herein.

Combination therapy can also embrace the administration of the anti-cancer therapeutic agent (e.g., an antibody) in further combination with other biologically active ingredients (e.g., a vitamin) and non-drug therapies (e.g., surgery or radiotherapy).

It should be appreciated that any combination of anti-cancer therapeutic agents may be used in any sequence for treating a cancer. The combinations described herein may be selected on the basis of a number of factors, which include but are not limited to reducing tumor formation or tumor growth, and/or alleviating at least one symptom associated with the cancer, or the effectiveness for mitigating the side effects of another agent of the combination. For example, a combined therapy as provided herein may reduce any of the side effects associated with each individual members of the combination, for example, a side effect associated with an administered anti-cancer agent.

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for determining tumor mutational burden (TMB) of a tumor sample previously obtained from a subject, the method comprising:
using at least one computer hardware processor to perform:
obtaining sequence reads, the sequence reads having been previously obtained by sequencing the tumor sample;
aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges;
identifying, using results of aligning the sequence reads to the population-specific genomic reference graph, a plurality of somatic variants; and
determining the TMB of the tumor sample using the identified plurality of somatic variants.

2. The method of claim 1, further comprising:
determining, using the determined TMB, to administer an immunotherapy to the subject.

3. The method of claim 2, wherein determining to administer the immunotherapy to the subject comprises:
determining whether the determined TMB is greater than or equal to a threshold TMB; and
determining to administer the immunotherapy to the subject after determining that the determined TMB is greater than or equal to the threshold TMB.

4. The method of claim 3,
wherein the sequence reads were previously-obtained by sequencing the tumor sample using whole exome sequencing, and
wherein the threshold TMB is between 150 variants/megabase (Mb) and 200 variants/Mb.

5. The method of claim 3,
wherein the sequence reads were previously obtained by sequencing the tumor sample using whole genome sequencing, and
wherein the threshold TMB is between 5 variants/Mb and 15 variants/Mb.

6. The method of claim 2, further comprising:
administering the immunotherapy to the subject.

7. The method of claim 2, wherein the immunotherapy is an immune checkpoint inhibitor.

8. The method of claim 7, wherein the immune checkpoint inhibitor is pembrolizumab.

9. The method of claim 1, wherein determining the TMB of the tumor sample comprises:
determining a number of somatic variants included in the identified plurality of somatic variants;
determining a size of a genomic region sequenced during the sequencing of the tumor sample; and
determining a ratio of the number of somatic variants to the size of the genomic region sequenced during the sequencing of the tumor sample.

10. The method of claim 1, wherein identifying the plurality of somatic variants comprises:
identifying a plurality of candidate variants using results of aligning the sequence reads to the population-specific genomic reference graph; and
filtering the plurality of candidate variants using at least a portion of the population-specific genomic reference graph to obtain the plurality of somatic variants.

11. The method of claim 10, wherein filtering the plurality of candidate variants using at least the portion of the population-specific genomic reference graph to obtain the plurality of somatic variants comprises:
identifying, using at least the portion of the population-specific genomic reference graph, one or more germline variants from among the plurality of candidate variants; and
excluding the one or more germline variants from the plurality of somatic variants.

12. The method of claim 1, wherein the results of aligning the sequence reads to the population-specific genomic reference graph comprise a plurality of aligned sequence reads, and wherein identifying the plurality of somatic variants comprises:
providing, as input to a somatic variant caller, the plurality of the aligned sequence reads and at least a portion of the population-specific genomic reference graph; and
obtaining, as output from the somatic variant caller, the plurality of somatic variants.

13. The method of claim 1, further comprising generating the population-specific genomic reference graph, the generating comprising:
obtaining an initial genomic reference, the initial genomic reference including the linear reference sequence; and
augmenting the initial genomic reference with the population-specific variants.

14. The method of claim 13, wherein augmenting the initial genomic reference with the population-specific variants comprises augmenting the initial genomic reference with one or more nodes and one or more edges, the one or more nodes and the one or more edges representing at least some of the population-specific variants.

15. The method of claim 1, wherein the population-specific genomic reference graph represents at least 10,000,000 nucleotides, at least 50,000,000 nucleotides, at least 100,000,000 nucleotides, at least 150,000,000 nucleotides, at least 200,000,000 nucleotides, or at least 250,000,000 nucleotides.

16. The method of claim 1,
wherein the nodes representing nucleotide sequences stored as respective strings of one or more symbols, and the edges including an edge representing a connection between at least two of the nodes.

17. The method of claim 1, wherein the at least one data structure comprises objects representing the nodes and pointers representing the edges, the objects comprising a first object representing a first node of the nodes, the first object storing at least one pointer representing at least one edge in the population-specific genomic reference graph from the first node to at least one other node.

18. The method of claim 1, further comprising sequencing the tumor sample to obtain the sequence reads.

19. A system, comprising:
at least one computer hardware processor; and
at least one non-transitory computer-readable storage medium storing processor-executable instructions that, when executed by the at least one computer hardware processor, cause the at least one computer hardware processor to perform a method for determining tumor mutational burden (TMB) of a tumor sample previously obtained from a subject, the method comprising:
obtaining sequence reads, the sequence reads having been previously obtained by sequencing the tumor sample;
aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges;
identifying, using results of aligning the sequence reads to the population-specific genomic reference graph, a plurality of somatic variants; and
determining the TMB of the tumor sample using the identified plurality of somatic variants.

20. At least one non-transitory computer-readable storage medium storing processor-executable instructions that, when executed by at least one computer hardware processor, cause the at least one computer hardware processor to perform a method for determining tumor mutational burden (TMB) of a tumor sample previously obtained from a subject, the method comprising:
obtaining sequence reads, the sequence reads having been previously obtained by sequencing the tumor sample;
aligning the sequence reads to a population-specific genomic reference graph by using at least one data structure representing the population-specific genomic reference graph, the population-specific genomic reference graph representing a linear reference sequence and population-specific variants relative to the linear reference sequence, the population-specific variants being associated with at least one population to which the subject belongs, the population-specific genomic reference graph comprising nodes and edges connecting the nodes, the at least one data structure storing data specifying the nodes and the edges;
identifying, using results of aligning the sequence reads to the population-specific genomic reference graph, a plurality of somatic variants; and
determining the TMB of the tumor sample using the identified plurality of somatic variants.
